(19) United States
(12) Patent Application Publication
(10) Pub. No.: US 2020/0375474 A1
(43) Pub. Date: Dec. 3, 2020

(54) ANALYSING ELECTROCARDIOGRAM DATA FROM A REMOTE PORTABLE SENSOR DEVICE

(71) Applicant: COALA-LIFE AB, Stockholm (SE)

(72) Inventors: Philip SIBERG, Stockholm (SE); Magnus SAMUELSSON, Stockholm (SE)

(21) Appl. No.: 16/960,493

(22) PCT Filed: Jan. 24, 2019
(86) PCT No.: PCT/SE2019/050051

§ 371 Date: Jul. 7, 2020

(30) Foreign Application Priority Data
Jan. 25, 2018 (SE) 1850082-7

(51) Int. Cl. A61B 5/0245; A61B 5/024; A61B 5/0404; A61B 5/0452; A61B 5/00

(52) U.S. Cl. CPC A61B 5/0245; A61B 5/02438; A61B 5/0404; A61B 2560/0431; A61B 5/6826; A61B 5/6823; A61B 5/0452

(57) ABSTRACT

It is provided a method for analysing heart data of a user. The method is performed in an analysis device and comprises the steps of: obtaining, from the portable sensor device, first electrocardiogram data, based on electrical signals measured by electrodes placed on the torso of the user; obtaining, from the portable sensor device, second electrocardiogram data, based on electrical signals measured by electrodes placed on two separate arms of the user; evaluating the first electrocardiogram data to determine whether there are any first abnormalities; evaluating the second electrocardiogram data to determine whether there are any second abnormalities; and determining that the heart is considered to need further examination only when there are both first abnormalities and second abnormalities.

### TECHNICAL FIELD

The invention relates to a method, an analysis device, a computer program and a computer program product analysing electrocardiogram data from a remote portable sensor device.

### BACKGROUND

ECG is an established technology where electric signals generated by the body of a patient are measured and analysed. Traditionally, a number of electrodes are placed on the body at various places. A conductive gel is used to provide better conductive contact between the electrode and the skin. The patient typically lies down for several minutes when the ECG is taken. The data detected using the electrodes is recorded and can be analysed by a professional, such as a physician or trained nurse. Once the measurement procedure is done, the conductive gel is wiped off.

While having proved useful, the traditional way of obtaining an ECG is not optimal in all cases. For instance, such an ECG needs to be measured in a clinic and the procedure is messy for the patient.

Lately, portable sensor devices with integral electrodes for obtaining ECG data have been developed. These portable sensor devices allow users to capture ECG data at will and also without the use of conductive gel. This gives the user greater control over when to capture ECG data and also in a much more convenient and less messy way.

The ECG data can be used to classify the patient in one of two states. A first state is a normal state, where nothing more needs to be done. A second state is a state where further investigation is needed.

However, the classification of the patient in one of the two states needs to be carefully balanced, such that serious heart conditions are not missed and such that further investigation is not recommended unnecessarily, causing stress and inconvenience for the patient.

### SUMMARY

It is an object to improve the classification accuracy of heart electrocardiogram (ECG) data.

According to a first aspect, it is provided a method for analysing heart data of a user. The method is performed in an analysis device and comprises the steps of: obtaining, from the portable sensor device, first electrocardiogram data, based on electrical signals measured by electrodes placed on the torso of the user; obtaining, from the portable sensor device, second electrocardiogram data, based on electrical signals measured by electrodes placed on two separate arms of the user; evaluating the first electrocardiogram data to determine whether there are any first abnormalities; evaluating the second electrocardiogram data to determine whether there are any second abnormalities; and determining that the heart is considered to need further examination only when there are both first abnormalities and second abnormalities.

The first electrocardiogram data may cover a different measurement time period than the second electrocardiogram data.

The second electrocardiogram data may be based on electrical signals measured by electrodes placed on two separate dexterities of the user.

The step of evaluating the first electrocardiogram data may comprise determining whether there are any first abnormalities based on heartbeat frequency of the first electrocardiogram data; and the step of evaluating the second electrocardiogram data may comprise to determining whether there are any second abnormalities based on heartbeat frequency of the second electrocardiogram data.

According to a second aspect, it is provided an analysis device for analysing heart data of a user. The analysis device comprises: a processor; and a memory storing instructions that, when executed by the processor, cause the analysis device to: obtain, from the portable sensor device, first electrocardiogram data, based on electrical signals measured by electrodes placed on the torso of the user; obtain, from the portable sensor device, second electrocardiogram data, based on electrical signals measured by electrodes placed on two separate arms of the user; evaluate first electrocardiogram data to determine whether there are any first abnormalities; evaluate the second electrocardiogram data to determine whether there are any second abnormalities; and determine that the heart is considered to need further examination only when there are both first electrocardiogram abnormalities and second abnormalities.

The first electrocardiogram data may cover a different measurement time period than the second electrocardiogram data.

The second electrocardiogram data may be based on electrical signals measured by electrodes placed on two separate dexterities of the user.

The instructions to evaluate the first electrocardiogram data may comprise instructions that, when executed by the processor, cause the analysis device to determine whether there are any first abnormalities based on heartbeat frequency of the first electrocardiogram data; and the instructions to evaluate the second electrocardiogram data comprise instructions that, when executed by the processor, cause the analysis device to determine whether there are any second abnormalities based on heartbeat frequency of the second electrocardiogram data.

According to a third aspect, it is provided a computer program for analysing heart data of a user. The computer program comprising computer program code which, when run on an analysis device causes the analysis device to: obtain, from the portable sensor device, first electrocardiogram data, based on electrical signals measured by electrodes placed on the torso of the user; obtain, from the portable sensor device, second electrocardiogram data, based on electrical signals measured by electrodes placed on two separate arms of the user; evaluate the first electrocardiogram data to determine whether there are any first abnormalities; evaluate the second electrocardiogram data to determine whether there are any second abnormalities; and determine that the heart is considered to need further examination only when there are both first abnormalities and second abnormalities.

According to a fourth aspect, it is provided a computer program product comprising a computer program according to the third aspect and a computer readable means on which the computer program is stored.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

The invention is now described, by way of example, with reference to the accompanying drawings, in which:

FIGS. 1A-B are schematic diagrams illustrating an environment in which embodiments presented herein can be applied;

FIG. 2 is a schematic diagram illustrating when the portable sensor device is used to capture measurements for ECG;

FIGS. 3A-B are schematic diagrams of views illustrating a physical representation of the portable sensor device according to one embodiment;

FIG. 4 is a schematic diagram illustrating the analysis device of FIGS. 1A-B according to one embodiment;

FIG. 5 is a flow chart illustrating embodiments of methods for analysing heart data of a user, the methods being performed in the analysis device of FIG. 1; and

FIG. 6 shows one example of a computer program product comprising computer readable means.

### DETAILED DESCRIPTION

The invention will now be described more fully hereinafter with reference to the accompanying drawings, in which certain embodiments of the invention are shown. This invention may, however, be embodied in many different forms and should not be construed as limited to the embodiments set forth herein; rather, these embodiments are provided by way of example so that this disclosure will be thorough and complete, and will fully convey the scope of the invention to those skilled in the art. Like numbers refer to like elements throughout the description.

According to embodiments herein, a portable device is used to capture both ECG data from both a torso measurement and a measurement between arms, e.g. on hands. The heart is considered to need further examination only when there are both ECG data sets indicate abnormalities. In this way, many false positive further examination considerations are avoided.

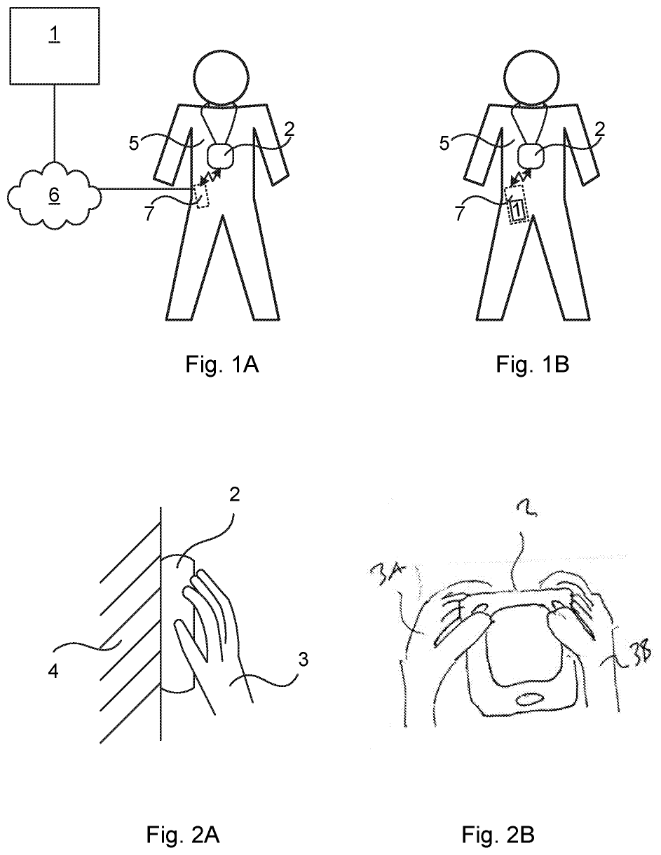

FIGS. 1A-B are schematic diagrams illustrating an environment in which embodiments presented herein can be applied.

Looking first to FIG. 1A, it is here shown a user 5 carrying a portable sensor device 2 in a necklace strap. The portable sensor device can be carried in any other way, e.g. in a pocket or in a handbag. The user 5 also carries a smartphone 7 e.g. in a pocket. The portable sensor device 2 and the smartphone 7 can communicate over any suitable wireless interface, e.g. using Bluetooth or Bluetooth Low Energy (BLE), ZigBee, any of the IEEE 802.11x standards (also known as WiFi), etc.

The smartphone 7 is also connected to a wide area network 6, such as the Internet, e.g. via WiFi or a cellular network, to allow communication with an analysis device 1, here in the form of a server. The portable sensor device 2 captures ECG (electrocardiogram) data and optionally also PCG (phonocardiogram) data and sends this data, via the smartphone 7, to the analysis device 1. The ECG data can be captured in (at least) two ways as shown in FIGS. 2A-B and explained below. This allows the analysis device 1 to determine whether the heart of the user 5 can be considered to be in a normal state or whether the heart needs further examination based on the ECG data captured in two different ways by the portable sensor device 2. Further investigation can be determined to be needed e.g. if any abnormal heart condition cannot be ruled out. It is to be noted that even if further investigation is to be performed, the heart can in fact be normal, i.e. non-pathological.

In FIG. 1B, the smartphone 7 contains the analysis device 1. In this way, the analysis can be performed locally, without the need for immediate access to the wide area network.

Alternatively, the analysis device can form part of the portable sensor device 2 (not shown). In such a case, the portable sensor 2 can also perform the functions of the smartphone 7.

FIGS. 2A-B are schematic diagrams illustrating when the portable sensor device 2 of FIG. 1 is used in two different ways to capture measurements for ECG.

In FIG. 2A, the portable sensor device 2 is placed on the skin of the torso of the user 5, close to the heart of the user. The user holds the portable sensor device 2 in place using a hand 3. This allows ECG measurements to take place locally, close to the heart. The ECG measurement on the torso has a low noise component since the measurement is close to the heart, whereby other muscle movements do not influence the ECG measurement much. This allows the P wave, representing atrial depolarization, to be easier detected, since the P wave has relatively low amplitude and is susceptible to noise.

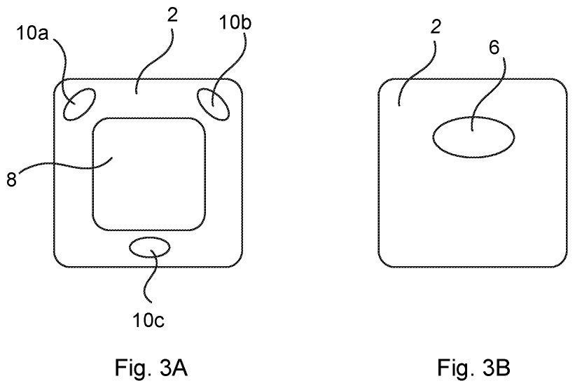

In FIG. 2B, the portable sensor device 2 is held by two hands 3a-b in an alternative way (compared to FIG. 2A) to obtain ECG measurements. The user holds the portable device 2 such that two skin on two respective arms, e.g. on respective dexterities (e.g. thumbs) from the hands 3a-b are placed in contact with electrodes of the portable sensor device 2, allowing ECG measurement. It is to be noted that there are no loose electrodes for the ECG measurement. Instead, the electrodes (as shown in FIG. 3A and described below) are provided integral to the portable sensor device 2. Hence, the measurement for the ECG is captured simply by the user holding the portable sensor device 2 in his/her hands to provide contact with the hands. When ECG measurements are performed from the dexterities, the ECG measurement is more standardised, i.e. the measurements are similar between different individuals. This measurement crosses the shoulders and form part of what is known as Einthoven lead 1. However, there may be noise forming part of the measurement due to muscle movement, e.g. in the arms.

When the ECG is measured from dexterities, the ECG measurement and the ECG measurement occur for different time periods.

FIGS. 3A-3B are schematic diagrams of views illustrating a physical representation of the portable sensor device 2 of FIG. 1 according to one embodiment.

In FIG. 3A, a bottom view of the portable sensor device 2 is shown. There are a first electrode 10a, a second electrode 10b and a third electrode 10c. In order to capture the ECG data, the electrodes 10a-c are placed on the casing of the portable sensor device 2 such that the user is able to bring at least two of the electrodes 10a-c in contact with the skin. It is to be noted that the portable sensor device 2 could also be provided with two electrodes, four electrodes or any other suitable number of electrodes. Using the electrodes, one or more analogue ECG signals are captured. The analogue ECG signals are converted to digital ECG signals using an analogue to digital (A/D) converter. The digital ECG signal is then sent to the analysis device for analysis.

Additionally, a transducer 8, e.g. in the form of a microphone, can be provided to convert sound captured by the body into electric analogue PCG signals. The analogue PCG signals are converted to digital PCG signals using an A/D converter. The digital PCG signal can also be sent to the analysis device for analysis together with the ECG signal

In FIG. 3B, a top view of the portable sensor device 2 is shown. Here, a user interface element 4 in form of a push button is shown. The push button can e.g. be used by the user to indicate when to start a measurement of ECG data and/or PCG data. It is to be noted that other user interface elements can be provided (not shown), e.g. more push buttons, Light Emitting Diodes (LEDs), a display, a speaker, a user microphone, accelerometer to detect motion, etc.

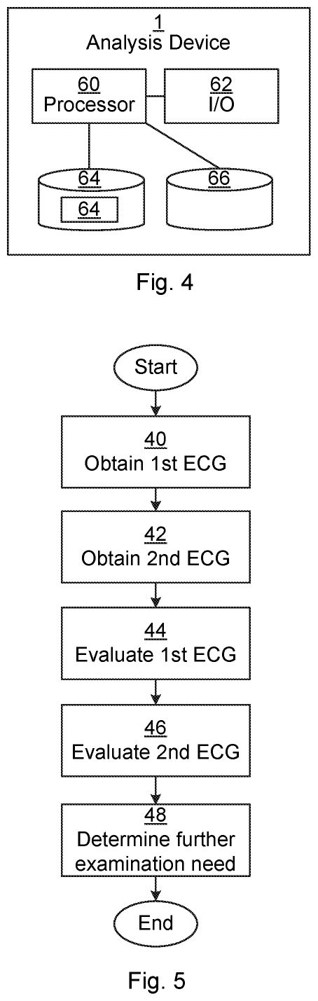

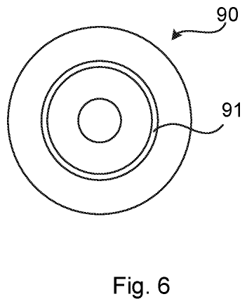

FIG. 4 is a schematic diagram illustrating the analysis device 1 of FIG. 1 according to one embodiment. As shown in FIGS. 1A-B, the analysis device can be implemented as part of a server or as part of a user device, such as a smartphone or alternatively as part of the portable sensor device. A processor 60 is provided using any combination of one or more of a suitable central processing unit (CPU), multiprocessor, microcontroller, digital signal processor (DSP), application specific integrated circuit etc., capable of executing software instructions 67 stored in a memory 64, which can thus be a computer program product. The processor 60 can be configured to execute the method described with reference to FIGS. 6A-B below.

The memory 64 can be any combination of read and write memory (RAM) and read only memory (ROM). The memory 64 also comprises persistent storage, which, for example, can be any single one or combination of magnetic memory, optical memory, solid state memory or even remotely mounted memory.

A data memory 66 is also provided for reading and/or storing data during execution of software instructions in the processor 60. The data memory 66 can be any combination of read and write memory (RAM) and read only memory (ROM).

The analysis device 1 further comprises an I/O interface 62 for communicating with other external entities, such as the smartphone 7 of the user using Internet Protocol (IP) over the wide area network 6.

Other components of the analysis device are omitted in order not to obscure the concepts presented herein

FIG. 5 is a flow chart illustrating embodiments of methods for analysing heart data of a user, the methods being performed in the analysis device of FIG. 1. It to be noted that the use of terms ‘first’ and ‘second’ herein is not used to denote any order or priority. These terms are merely used as labels to allow reference to different instances of e.g. electrocardiogram data and abnormalities.

In an obtain first ECG step 40, first electrocardiogram data, based on electrical signals measured by electrodes placed on the torso of the user, is obtained from a portable sensor device.

In an obtain second ECG step 42, second ECG data, based on electrical signals measured by electrodes placed on two separate arms of the user, is obtained from the portable sensor device.

As explained above, the ECG data is based on electrical signals measured by electrodes placed on the body of the user. The ECG data can be the digital ECG data described above. The electrocardiogram data is received from the portable measurement device

It is to be noted that the order in which steps 40 and 42 are performed is not important.

In an evaluate first ECG step 44, the first ECG data is evaluated to determine whether there are any first abnormalities. Abnormal, whenever used in the description or claims, is here to be construed as a condition where a pathological condition cannot be ruled out with the currently available information. A further evaluation can later result in a conclusion that the heart in fact is normal (non-pathological). For the first ECG data, captured from the torso, the data can be evaluated e.g. by evaluating if the P wave is weak or non-detectable. Also, heart beat irregularity can be evaluated to find abnormalities.

In an evaluate second ECG step 46, the second ECG data is evaluated to determine whether there are any second abnormalities. For the second ECG data, captured across the shoulders, the data can be evaluated using Einthoven lead 1 methods known in the art per se. Also, heart beat irregularity can be evaluated to find abnormalities.

It is to be noted that the order in which steps 44 and 46 are performed is not important.

In a determine further examination need step 48, the analysis device determines that the heart is considered to need further examination only when there are both first abnormalities and second abnormalities. In other words, the analysis device classified the patient in one of two states. A first state is a normal state, where nothing more needs to be done. A second state is a state where further investigation is needed. For instance, irregular heart beat frequency can be determined to be abnormal in both ECG data, such as an extra heartbeat, too high frequency or too low frequency.

In this way, many false positives (of determining further examination) are avoided. For instance, the user may have just come home from being outside and may be physically strained, e.g. from walking up steps to the home. In such a situation, the first measurement can be less reliable for determining further examination and the second measurement is more reliable, whereby it is likely incorrect to indicate further examination if the first measurement is abnormal and the second shows no abnormality. In another example, the user can be nervous when performing a first measurement and calms down for the second measurement, or vice versa. In these situations, it does not matter if the ECG measurement taken first is from two separate arms and the ECG measurement taken subsequently is from the torso, or vice versa.

Hence, when the first ECG data covers a different measurement time period than the second ECG data, this determination allows for a more reliable determination of a need for further examination.

One might be inclined to envisage that the further examination determination of this method is too negligent and might result in many missed abnormal heart conditions (i.e. false negatives). However, the inventors have found in practical tests that this is not the case and that this method is very well balanced between not providing too many false positives nor too many false negatives. This provides a simple way to increase accuracy in classification in when the heart is considered to need further examination.

FIG. 6 shows one example of a computer program product comprising computer readable means. On this computer readable means a computer program 91 can be stored, which computer program can cause a processor to execute a method according to embodiments described herein. In this example, the computer program product is an optical disc, such as a CD (compact disc) or a DVD (digital versatile disc) or a Blu-Ray disc. As explained above, the computer program product could also be embodied in a memory of a device, such as the computer program product 64 of FIG. 4. While the computer program 91 is here schematically shown as a track on the depicted optical disk, the computer program can be stored in any way which is suitable for the computer program product, such as a removable solid state memory, e.g. a Universal Serial Bus (USB) drive.

The invention has mainly been described above with reference to a few embodiments. However, as is readily appreciated by a person skilled in the art, other embodiments than the ones disclosed above are equally possible within the scope of the invention, as defined by the appended patent claims.

What is claimed is:

1. A method for analysing heart data of a user, the method being performed in an analysis device and comprising:
obtaining, from the portable sensor device first electrocardiogram data, based on electrical signals measured by electrodes placed on the torso of the user;
obtaining, from the portable sensor device second electrocardiogram data, based on electrical signals measured by electrodes placed on two separate arms of the user;
evaluating the first electrocardiogram data to determine whether there are any first abnormalities;
evaluating the second electrocardiogram data to determine whether there are any second abnormalities; and
determining that the heart is considered to need further examination only when there are both first abnormalities and second abnormalities.

2. The method according to claim 1, wherein the first electrocardiogram data covers a different measurement time period than the second electrocardiogram data.

3. The method according to claim 1, or wherein the second electrocardiogram data is based on electrical signals measured by electrodes placed on two separate thumbs of the user.

4. The method according to claim 1, wherein the step of evaluating the first electrocardiogram data comprises determining whether there are any first abnormalities based on heartbeat frequency of the first electrocardiogram data; and the step of evaluating the second electrocardiogram data comprises to determining whether there are any second abnormalities based on heartbeat frequency of the second electrocardiogram data.

5. An analysis device for analysing heart data of a user, the analysis device comprising:
a processor; and
a memory storing instructions that, when executed by the processor, cause the analysis device to:
obtain, from the portable sensor device, first electrocardiogram data, based on electrical signals measured by electrodes placed on the torso of the user;
obtain, from the portable sensor device, second electrocardiogram data, based on electrical signals measured by electrodes placed on two separate arms of the user;
evaluate first electrocardiogram data to determine whether there are any first abnormalities;
evaluate the second electrocardiogram data to determine whether there are any second abnormalities; and
determine that the heart is considered to need further examination only when there are both first electrocardiogram abnormalities and second abnormalities.

6. The analysis device according to claim 5, wherein the first electrocardiogram data covers a different measurement time period than the second electrocardiogram data.

7. The analysis device according to claim 5, wherein the second electrocardiogram data is based on electrical signals measured by electrodes placed on two separate thumbs of the user.

8. The analysis device according to claim 5, wherein the instructions to evaluate the first electrocardiogram data comprise instructions that, when executed by the processor, cause the analysis device to determine whether there are any first abnormalities based on heartbeat frequency of the first electrocardiogram data; and the instructions to evaluate the second electrocardiogram data comprise instructions that, when executed by the processor, cause the analysis device to determine whether there are any second abnormalities based on heartbeat frequency of the second electrocardiogram data.

9. A computer program for analysing heart data of a user, the computer program comprising computer program code which, when run on an analysis device causes the analysis device to:
obtain, from the portable sensor device, first electrocardiogram data, based on electrical signals measured by electrodes placed on the torso of the user;
obtain, from the portable sensor device, second electrocardiogram data, based on electrical signals measured by electrodes placed on two separate arms of the user;
evaluate the first electrocardiogram data to determine whether there are any first abnormalities;
evaluate the second electrocardiogram data to determine whether are any second abnormalities; and
determine that the heart is considered to need further examination only when there are both first abnormalities and second abnormalities.

10. A computer program product comprising a computer program according to claim 9 and a computer readable means on which the computer program is stored.
